(19) United States
(12) Patent Application Publication
(10) Pub. No.: US 2022/0193174 A1
(43) Pub. Date: Jun. 23, 2022

(54) NASAL SPRAY FOR PREVENTING INFLUENZA BASED ON RED PEPPER EXTRACT AND HEPARIN AND PREPARATION METHOD THEREOF

(71) Applicant: QUFU NORMAL UNIVERSITY, Shangdong (CN)

(72) Inventors: Ge Yang, Shangdong (CN); Yifei Liu, Shangdong (CN); Chengchuan Che, Shangdong (CN); Jinfeng Liu, Shangdong (CN); Zhijin Gong, Shangdong (CN); Yang Sun, Shangdong (CN); Qi Chen, Shangdong (CN)

(73) Assignee: QUFU NORMAL UNIVERSITY, Shangdong (CN)

(21) Appl. No.: 17/329,086

(22) Filed: May 24, 2021

(30) Foreign Application Priority Data
Dec. 18, 2020 (CN) 202011503902.9

(51) Int. Cl. A61K 36/81; A61K 9/00; A61K 9/12; A61K 47/36; A61K 47/10; A61K 31/727; A61P 31/16

(52) U.S. Cl. CPC A61K 36/81; A61K 9/0043; A61K 9/12; A61K 47/36; A61K 2236/51; A61K 31/727; A61P 31/16; A61K 2236/15; A61K 2236/333; A61K 47/10

(57) ABSTRACT

The present disclosure belongs to the technical field of medicine preparation, and particularly relates to a nasal spray for preventing influenza based on a red pepper extract and heparin and a preparation method thereof. A nasal spray consists of the following raw materials in parts by weight: 10% γ-PGA-CS, 5-10% glycerol, 2% red pepper extract, 5-10% heparin solution, 2-2.4% ethanol and balance of purified water. The present disclosure can not only inhibit germs and diminish inflammation, and has a better therapeutic effect on rhinitis, but also can keep the inner surface of a nasal cavity moist for a long time. Moreover, the present disclosure can reduce side effects on a human body by changing the type of bactericides in the nasal spray, thus keeping the nasal cavity clean and moist for a long time.

### CROSS-REFERENCE TO RELATED APPLICATION

This application claims the priority benefit of China Application No. 202011503902.9, filed on Dec. 18, 2020. The entirety of the above-mentioned patent application is hereby incorporated by reference herein and made a part of this specification.

### BACKGROUND

### Technical Field

The present disclosure belongs to the technical field of medicine preparation, and particularly relates to a nasal spray for preventing influenza based on a red pepper extract and heparin and a preparation method thereof.

### Description of Related Art

The importance of breathing to maintain our life is well known; and the nasal cavity is the first guard of our respiratory system. It is calculated that a normal adult breathes in and out 15000 liters of air in total 24 hours a day. People can choose drinking water that is required every day, but cannot choose air that is required to breathe every moment; and people inhale different qualities of air due to their different living environment. With the development of industrial production and transportation, there are more and more dust, smoke, organic toxicants, bacteria and other hazardous substances in the air; and moreover, the hazardous substances retained in the nasal vestibule of people get more and more. Dirt which cannot be removed in time, vibrissa incrustation, and retention and breeding of a large number of bacteria and viruses will stimulate the nasal mucosa to cause discomfort, which will increase the occurrence risk of diseases, such as, various acute and chronic rhinitis, nasosinusitis, allergic rhinitis and cold undoubtedly. Moreover, the formation of dry scab or caseous substances caused by the retention of viscous and purulent secretions in nasal cavity always block the nasal cavity to influence the air circulation of the nasal cavity; and the clinical symptoms are dryness, itching, nasal obstruction, fetor narium, dizziness, headache, insomnia, inappetence and the like. Therefore, the nasal cavity is cleaned to remove dry scab and caseous substances, recover clean and smooth nasal cavity, and keep the nasal cavity moist and normal olfactory functions, thus promoting the recovery of rhinitis sicca, atrophic rhinitis and caseous rhinitis. Nasal cavity cleaning can quickly remove the dirt from the nasal cavity to relieve allergic rhinitis and reduce the incidence rate of allergic rhinitis. The dry scab existing in the nasal vestibule will cause discomfort; if the cleaning method is improper, it not only can affect the personal image, but also easily cause the nasal vestibule skin injury and vibrissa falling off, even cause bleeding, infection and other consequences when serious; the nasal cavity cleaning can effectively improve the condition. Therefore, the frequent cleaning of nasal vestibule can not only keep the nasal cavity clean, but also can prevent the occurrence of various diseases. Nasal spray is one of preferred cleaning articles.

The current nasal spray aims at sterilization and disinfection, and it is mainly prepared by physiological saline and supplemented with a bactericide, such as aspirin, ibuprofen, chlorpheniramine maleate and other antibiotics. These nasal cleaning solutions can effectively kill most of germs to exert a certain sterilization and disinfection effects on nasal cavity, but it is neglected that the use of drugs and antibiotics in cleaning or sterilizing the nasal cavity will increase the side effects on human bodies and the harmful degree of medication. Moreover, since most of the products on the market are single in effect and short in the maintenance time of flushing effect on the nasal cavity during use, some patients with dry, itching and astringent nose often suffer a dry and itch state again just after spraying the spray into nasal cavity; and even an individual patient has to increase the use frequency of the spray due to work or other demands, which causes inconvenience to life, and increases the bearing pressure of the nasal cavity. The nasal patients increase the use frequency under the condition of not achieving the expected effect of the product, causing a lot of unnecessary troubles.

SUMMARY

Directed to the problems in the prior art, the present disclosure provides a nasal spray for preventing influenza based on a red pepper extract and heparin. The spray not only can sterilize germs and diminish inflammation, but also can keep the inner surface of a nasal cavity moist for a long time and is beneficial to human health.

The present disclosure further provides a preparation method of the nasal spray for preventing influenza based on the red pepper extract and the heparin; and the preparation method is simple in process, pollution-free, energy-saving and suitable for industrial implementation.

To achieve the above objectives, the technical solution adopted in the present disclosure is as follows:

The present disclosure provides a nasal spray for preventing influenza based on a red pepper extract and heparin, consisting of the following raw materials in parts by weight: 10% γ-PGA-CS, 5-10% glycerol, 2% red pepper extract, 5-10% heparin solution, 2-2.4% ethanol and balance of purified water.

The γ-PGA-CS used in the present disclosure is prepared by the following method of: dissolving chitosan in a 95% acetic acid solution, uniformly stirring at normal temperature and diluting with deionized water to prepare a 70% chitosan solution; dropwisely adding a 70% poly-γ-glutamic acid solution to an equal volume of 70% chitosan solution, magnetically stirring, fully mixing, and freeze-drying to obtain a poly-γ-glutamic acid/chitosan dry powder, γ-PGA-CS.

The red pepper extract used in the present disclosure is prepared by the following method of: drying red pepper at 40° C., and then crushing with a pulverizer, repeatedly extracting with ethanol till the red pepper is soaked by ethanol, and drying in vacuum to obtain the red pepper extract.

The present disclosure further provides a preparation method of the nasal spray, including the following steps of:

(1) dissolving the poly-γ-glutamic acid/chitosan dry powder γ-PGA-CS in water to obtain a 10 g/L solution A;

(2) dissolving the red pepper extract with 30% ethanol water to obtain a 3 g/L solution B;

(3) dissolving heparin in water to obtain a heparin solution, that is a 10 g/L solution C;

(4) mixing the solution A with the solution C, and placing into 1 L deionized purified water, stirring for 2-5 min by a magnetic stirrer, and adding glycerol to a mixed solution to prepare a solution D;

(5) taking the solution D and mixing with the solution B, oscillating for 3-5 min at a low frequency, and filtering by a filter to obtain a solution F;

(6) sterilizing the solution F, and can sealing for storage.

Preferably, the sterilization in this present disclosure is performed by irradiating an ultraviolet light source to disinfect and kill microorganisms in a solution. By changing the type of bactericides in the nasal spray, natural bactericidal and anti-inflammatory substances in a red pepper extract are used to replace traditional antibiotics, such as aspirin, ibuprofen, and chlorpheniramine maleate; under the synergistic effect of the red pepper extract and heparin, the nasal spray of this present disclosure can improve the sterilization performance, reduce side effects on human bodies; moreover, the germs in the nasal cavity are not resistant to the spray. The substances via glycerol and γ-PGA-CS form a layer of moisturizing film on the inner wall of the nasal cavity, so that the product improves the durability of cleaning and moistening the nasal cavity on the premise of eliminating germs in the nasal cavity, thus keeping the nasal cavity clean and moist for a long time. The present disclosure obviously improves the defect of the short maintenance time of the use effect of a traditional product, which not only improves the expected effect of the product, but also relieves the pressure of the patients during the use procedure of the nasal spray.

The present disclosure has the beneficial effects:

(1) The nasal spray prepared by the present disclosure has the advantages of inhibiting germs and diminishing inflammation, moisturizing for a long time, greatly reducing the use frequency and lowering side effects on human bodies; moreover, and the germs in the nasal cavity have no drug resistance.

(2) The nasal spray prepared by the present disclosure can prevent influenza, has good universality and is free of stimulation.

(3) The preparation method provided by the present disclosure is simple in process, pollution-free, energy-saving and suitable for industrial implementation.

### DESCRIPTION OF THE EMBODIMENTS

The present disclosure will be further described in detail below to enable those skilled in the art to implement the present disclosure with reference to the description.

### EXAMPLE 1

A nasal spray, included the following components in percentage by weight: 10% γ-PGA-CS, 5% glycerol, 2% red pepper extract, 5% heparin solution, 2% ethanol, and balance of purified water.

A preparation method of a nasal spray, including the following steps of:

step 1: chitosan was dissolved in a 95% acetic acid solution, uniformly stirred at normal temperature and diluted with deionized water to prepare a 70% chitosan solution; a 70% poly-γ-glutamic acid solution was dropwisely added to an equal volume of 70% chitosan solution, magnetically stirred, fully mixed, and frozen-dried to obtain a poly-γ-glutamic acid/chitosan dry powder; where the dry powder could be dissolved in water to obtain a 10 g/L solution A;

step 2: 50 g red pepper were dried at 40° C., crushed with a pulverizer, repeatedly leached with ethanol, dried in vacuum to obtain a red pepper extract, and then the red pepper extract was dissolved with 30% ethanol water to obtain a 3 g/L solution B;

step 3: heparin was dissolved in water to obtain a heparin solution, that is a 10 g/L solution C;

step 4: the solution A and solution C were mixed, and placed into 1 L deionized purified water, stirred for 2-5 min with a magnetic stirrer, and glycerol was added to the mixed solution to prepare a solution D;

step 5: the solution D was taken and mixed with the solution B, oscillated for 3-5 min at a low frequency, and filtered by a filter to obtain a solution F;

step 6: the solution obtained in step (5) was irradiated by an ultraviolet light source, and can sealed for storage.

### EXAMPLE 2

A nasal spray, included the following components in percentage by weight: 10% γ-PGA-CS, 7% glycerol, 2% red pepper extract, 8% heparin solution, 2% ethanol, and balance of purified water.

The preparation method was the same as that of Example 1.

### EXAMPLE 3

A nasal spray, included the following components in percentage by weight: 10% γ-PGA-CS, 10% glycerol, 2% red pepper extract, 10% heparin, 2% ethanol, and balance of purified water.

The preparation method was the same as that of Example 1.

### Comparative Example 1

A commercially available physiological seawater spray.

### Comparative Example 2

A nasal spray, included the following components in percentage by weight: 10% γ-PGA-CS, 5% glycerol, 7% red pepper extract, 2% ethanol, and balance of purified water.

The preparation method was as follows:

step 1: chitosan was dissolved in a 95% acetic acid solution, uniformly stirred at normal temperature and diluted with deionized water to prepare a chitosan solution; a 70% poly-γ-glutamic acid solution was dropwisely added to an equal volume of 70% chitosan solution, magnetically stirred, fully mixed, and frozen-dried to obtain a poly-γ-glutamic acid/chitosan dry powder; where the dry powder could be dissolved in water to obtain a 10 g/L solution A;

step 2: 50 g red pepper were dried at 40° C., crushed with a pulverizer, repeatedly leached with ethanol, dried in vacuum to obtain a red pepper extract, and then the red pepper extract was dissolved with 30% ethanol water to obtain a 3 g/L solution B;

step 3: the solution A was placed into 1 L deionized purified water, stirred for 2-5 min with a magnetic stirrer, and glycerol was added to the mixed solution to prepare a solution D;

step 4: the solution D was taken and mixed with the solution B, oscillated for 3-5 min at a low frequency, and filtered by a filter to obtain a solution F;

step 5: the solution obtained in step (5) was irradiated by an ultraviolet light source, and can sealed for storage.

### Comparative Example 3

A nasal spray, included the following components in percentage by weight: 10% γ-PGA-CS, 5% glycerol, 7% heparin solution, 2% ethanol, and balance of purified water.

The preparation method was as follows:

step 1: chitosan was dissolved in a 95% acetic acid solution, uniformly stirred at normal temperature and diluted with deionized water to prepare a chitosan solution; a 70% poly-γ-glutamic acid solution was dropwisely added to an equal volume of 70% chitosan solution, magnetically stirred, fully mixed, and frozen-dried to obtain a poly-γ-glutamic acid/chitosan dry powder; where the dry powder could be dissolved in water to obtain a 10 g/L solution A;

step 2: 50 g red pepper were dried at 40° C., crushed with a pulverizer, repeatedly leached with ethanol, dried in vacuum to obtain a red pepper extract, and then the red pepper extract was dissolved with 30% ethanol water to obtain a 3 g/L solution B;

step 3: heparin was dissolved in water to obtain a heparin solution, that is a 10 g/L solution C;

step 4: the solution A and solution C were mixed, and placed into 1 L deionized purified water, stirred for 2-5 min with a magnetic stirrer, and glycerol was added to the mixed solution to prepare a solution D;

step 5: the solution D was taken and oscillated for 3-5 min at a low frequency, and filtered by a filter to obtain a solution F;

step 6: the solution obtained in step (5) was sterilized and can sealed for storage.

(I) Bacteriostasis test

The nasal sprays prepared in Examples 1-3 and Comparative Examples 1-3 were subjected to antibacterial tests, and the specific test results were shown in Table 1.

TABLE 1
Inhibitory zone test of a nasal spray solution

Sample inhibitory zone diameter (mm)

| | Staphylococcus | Escherichia | Pseudomonas |
|---|---|---|---|
| Group | aureus | coli | aeruginosa |
| Example 1 | 8.50 | 6.75 | 5.80 |
| Example 2 | 8.80 | 6.50 | 6.54 |
| Example 3 | 8.35 | 7.20 | 6.85 |
| Comparative | 5.15 | 2.50 | 4.10 |
| Example 1 | | | |
| Comparative | 7.39 | 6.02 | 5.02 |
| Example 2 | | | |
| Comparative | 3.24 | 2.62 | 4.03 |
| Example 3 | | | |

Analysis of experimental results: the nasal spray solutions in Examples 1-3 had an inhibitory effect on common pathogenic bacteria in the nasal cavity; and there was a minor difference in effect; the nasal spray solution in Comparative Example 1 also had an inhibitory effect, but the inhibitory effect was poor; in Comparative Examples 2 and 3, due to a single active ingredient, the inhibitory effect was inferior to that of the present disclosure. It indicates that the present disclosure has better bactericidal and bacteriostatic effects.

(II) Clinical trails

The nasal sprays of Examples 1-3 served as treatment groups and the comparative example as a control group, 100 patients with dry itching rhinitis were tested: 50 males and 50 females, aged from 18 to 55 years old. 100 patients were randomly divided into two groups; 25 patients were randomly allocated to Examples 1-3 respectively in the treatment group, and 25 patients were randomly allocated to the control group; and there was no statistical significance in age, sex, condition and course of disease among the six groups (P>0.05).

1. Diagnostic method:

(1) Clinical symptoms: dry and burning nasal cavity and nasopharynx, itching in the nose, frequent sneezing, headache, hyposmia, and memory deterioration.

(2) Examination: under the examination of an anterior rhinoscope, the nasal mucosa was hyperemia and swelling, the inferior nasal concha was hyperemia and swelling, there were more secretions in meatus nasi communis or basis nasi; the secretions showed watery in initial stage, and gradually changed to pus, scab, and the like.

2. Therapeutic method:

Treatment group: nasal sprayers with the nasal sprays of Examples 1 to 3 were administered separately, and nasal sprays were sprayed against the inside of the nasal cavity by the nasal sprayers when patients felt uncomfortable and itchy in the nasal cavity.

Control group: nasal sprayers with the nasal sprays of Comparative Example 1 were administered; and nasal sprays were sprayed against the inside of the nasal cavity by the nasal sprayers when patients felt uncomfortable and itchy in the nasal cavity.

Course of treatment: for 7 days, the average number of uses per day was recorded and the therapeutic effect was examined.

Criteria for the therapeutic effect:

Recovery: the symptoms of nasal obstruction, more nasal discharge, headache and the like completely disappeared; the nasal cavity and passage were clean.

Marked effective: the symptoms disappeared basically, the nasal cavity was ventilated well and there was a little of mucus in the nasal passage.

Effective: the purulent nasal discharge was significantly reduced, the nasal obstruction was relieved, and intranasal pus was reduced.

Inefficient: the symptoms and signs were not changed.

3. Results

The results were as shown in Table 2.

TABLE 2
Clinical trial results

| | Average | | Marked | | Ineffi- |
|---|---|---|---|---|---|
| | number of | Recovery | effective | Effective | ciency |
| | use per day | rate (%) | rate (%) | rate (%) | (%) |
| Example 1 | 3 | 80 | 20 | 0 | 0 |
| Example 2 | 3 | 80 | 16 | 4 | 0 |
| Example 3 | 3 | 84 | 12 | 4 | 0 |
| Comparative | 6 | 60 | 20 | 12 | 8 |
| Example 1 | | | | | |

Analysis of Experimental Results

The use frequency of Comparative Example 1 was significantly higher than that of Examples 1-3; and the therapeutic effect of Examples 1-3 was higher than that of Comparative Example 1.

Clinical trial results: there is no case of infection and adverse reaction in the clinical verification; and the subject is free of adverse stimulation in the test procedure, indicating that the present disclosure has safety and efficacy in clinical use. Since the use effect in Examples 1-3 are basically the same; moreover with the increase of the content of glycerol, the solution gets more and more sticky, which is not beneficial for the spray to spray out; therefore, in view of use and economic aspects, Example 1 is selected as a preferred embodiment.

The above experiment indicates that the present disclosure not only can inhibit bacteria and diminish inflammation, and has a good therapeutic effect on rhinitis, but also can keep the inner surface of the nasal cavity moist for a long time; moreover, by changing the type of bactericides in the nasal spray, natural bactericidal and anti-inflammatory substances in a red pepper extract are used to replace traditional antibiotics, such as aspirin, ibuprofen, and chlorpheniramine maleate; under the synergistic effect with heparin, the nasal spray of this present disclosure can improve the efficacy, reduce side effects on human bodies; moreover, the germs in the nasal cavity are not resistant to the spray. The substances via glycerol and γ-PGA-CS form a layer of moisturizing film on the inner wall of the nasal cavity, so that the product improves the durability of cleaning and moistening the nasal cavity on the premise of eliminating germs in the nasal cavity, thus keeping the nasal cavity clean and moist for a long time. The present disclosure obviously improves the defect of short maintenance time of the use effect of the traditional product, which not only improves the expected effect of the product, but also relieves the pressure of the patients during the use procedure of the nasal spray.

What is claimed is:

1. A nasal spray for preventing influenza based on a red pepper extract and heparin, wherein the nasal spray consists of the following raw materials in parts by weight: 10% poly-γ-glutamic acid/chitosan dry powder, 5-10% glycerol, 2% red pepper extract, 5-10% heparin solution, 2-2.4% ethanol and balance of purified water.

2. The nasal spray according to claim 1, wherein the poly-γ-glutamic acid/chitosan dry powder is prepared by the following method of: dissolving chitosan in a 95% acetic acid solution, uniformly stirring at normal temperature and diluting with deionized water to prepare a 70% chitosan solution; dropwisely adding a 70% poly-γ-glutamic acid solution to an equal volume of 70% chitosan solution, magnetically stirring, fully mixing, and freeze-drying to obtain the poly-γ-glutamic acid/chitosan dry powder.

3. The nasal spray according to claim 1, wherein the red pepper extract is prepared by the following method of: drying red pepper at 40° C., and then crushing with a pulverizer, repeatedly extracting with ethanol till the red pepper is soaked by ethanol, and drying in vacuum to obtain the red pepper extract.

4. A preparation method of the nasal spray according to claim 1, comprising the following steps of:
(1) dissolving the poly-γ-glutamic acid/chitosan dry powder in water to obtain a 10 g/L solution A;
(2) dissolving the red pepper extract with 30% ethanol water to obtain a 3 g/L solution B;
(3) dissolving the heparin in water to obtain the heparin solution, that is a 10 g/L solution C;
(4) mixing the solution A with the solution C, and placing into 1 L deionized purified water, stirring for 2-5 min by a magnetic stirrer, and adding the glycerol to a mixed solution to prepare a solution D;
(5) taking the solution D and mixing with the solution B, oscillating for 3-5 min at a low frequency, and filtering by a filter to obtain a solution F;
(6) sterilizing the solution F, and can sealing for storage.

5. A preparation method of the nasal spray according to claim 2, comprising the following steps of:
(1) dissolving the poly-γ-glutamic acid/chitosan dry powder in water to obtain a 10 g/L solution A;
(2) dissolving the red pepper extract with 30% ethanol water to obtain a 3 g/L solution B;
(3) dissolving the heparin in water to obtain the heparin solution, that is a 10 g/L solution C;
(4) mixing the solution A with the solution C, and placing into 1 L deionized purified water, stirring for 2-5 min by a magnetic stirrer, and adding the glycerol to a mixed solution to prepare a solution D;
(5) taking the solution D and mixing with the solution B, oscillating for 3-5 min at a low frequency, and filtering by a filter to obtain a solution F;
(6) sterilizing the solution F, and can sealing for storage.

6. A preparation method of the nasal spray according to claim 3, comprising the following steps of:
(1) dissolving the poly-γ-glutamic acid/chitosan dry powder in water to obtain a 10 g/L solution A;
(2) dissolving the red pepper extract with 30% ethanol water to obtain a 3 g/L solution B;
(3) dissolving the heparin in water to obtain the heparin solution, that is a 10 g/L solution C;
(4) mixing the solution A with the solution C, and placing into 1 L deionized purified water, stirring for 2-5 min by a magnetic stirrer, and adding the glycerol to a mixed solution to prepare a solution D;
(5) taking the solution D and mixing with the solution B, oscillating for 3-5 min at a low frequency, and filtering by a filter to obtain a solution F;
(6) sterilizing the solution F, and can sealing for storage.
